(19) United States
(12) Patent Application Publication
(10) Pub. No.: US 2017/0319464 A1
(43) Pub. Date: Nov. 9, 2017

(54) FRAGRANT NAIL POLISH

(71) Applicant: PURE EUPHORIA PTY LTD, Peregian Beach, Queensland (AU)

(72) Inventor: Jackie Wright, Peregian Beach, Queensland (AU)

(73) Assignee: PURE EUPHORIA PTY LTD, Peregian Beach, Queensland (AU)

(21) Appl. No.: 15/528,410

(22) PCT Filed: Nov. 19, 2015
(86) PCT No.: PCT/AU2015/050727

§ 371 Date: May 19, 2017

(30) Foreign Application Priority Data
Nov. 19, 2014 (AU) 2014904652

(51) Int. Cl. A61K 8/92; A61K 8/81; A61K 8/37; A61K 8/34; A61K 8/73; A61K 8/41; A61Q 3/02

(52) U.S. Cl. CPC A61K 8/922; A61Q 3/02; A61K 8/8129; A61K 8/8147; A61K 8/34; A61K 8/731; A61K 8/416; A61K 8/37; A61K 2800/52

(57) ABSTRACT

The present invention provides a fragrant nail polish. The fragrant nail polish is made fragrant by essential oil. The film-former, solvents and resins in the polish are stabilised by stabilising agents.

### FIELD OF THE INVENTION

The present invention relates to fragrant nail polish, in particular nail polish made fragrant by essential oil.

### BACKGROUND OF THE INVENTION

Nail polish is applied to finger and toe nails to adjust their cosmetic appearance. A good nail polish applies evenly and easily, dries quickly, has high physical and chemical stability for a long shelf life, sets to a hard and smooth surface with uniform thickness, high lustre, excellent adhesion to the nail and good elasticity to prevent chipping and peeling of the polish. A good nail polish is also resistant to light, water and detergents so the polish remains adhered to the nail for as long as possible following application. A good cosmetic nail polish is also harmless to skin and nails. Cosmetic nail polishes are expected to last for several days to several weeks following application.

Medical nail varnishes have also been developed, for example, WO 2012/035478 describes an antifungal varnish composition. The antifungal varnish composition is for stripping nails, ie causing loss of an infected nail. The varnish comprises a liquid nail lacquer, an essential oil and a keratolytic agent. Medical nail varnishes are usually not appropriate for cosmetic use.

Traditional nail polish has an unpleasant ‘chemical’ odour. There is a need for development of nail polishes with a pleasant aroma.

One difficulty with development of pleasantly aromatic nail polish is that additives such as fragrance can destabilise the nail polish resulting in one or more of chemical instability in the nail polish composition, discolouration in both transparent and clear polish, prevention of nail polish hardening, increased chipping of the nail polish, and shortened effective life of the nail polish. The quantity of fragrance required to overpower the ‘chemical’ odour of the nail polish and create a pleasant aroma for many nail polish formulations is high. This often means that an aroma can only be detected only at a concentration of fragrance that negatively impacts the performance of the nail polish, for example, by unacceptably lessening the speed of drying, the evenness of spread, the hardness or the smoothness of the polish or the shelf life of the product, for example, the length of time for which the liquid formulation remains homogenous. Thus, there is a need for a fragrant nail polish with that retains the features of a good cosmetic nail polish.

There is also a need for development of 3-free and 5-free nail polish with a pleasant aroma.

There is also consumer demand for a nail polish with a natural fragrance. The composition of a natural fragrance is more variable than that of a synthetic fragrance. For example, essential oils contain a very complex mix of compounds that depends on their method of preparation, the species of plant from which there are extracted, where that plant was grown, the season of the extraction and many other factors. In addition, the scent of an essential oil is often less potent than for a synthetic fragrance so a greater quantity of an essential oil may be required to enable detection of the aroma of an essential oil than the comparative quantity required to detect a synthetic fragrance. Therefore, the difficulty that the concentration of fragrance needed to detect the aroma and for the aroma to overpower the ‘chemical’ fragrance of the other ingredients of the nail polish is a quantity that negatively impacts performance of the polish is often greater when the fragrance is provided by an essential oil. In addition, the many compounds in an essential oil increases the risk of a chemical interaction with the nail polish formulation that detracts from the qualities of a good cosmetic nail polish. For these reasons, preparation of a nail polish made fragrant with essential oil has been considered impossible.

WO 2009/094348 describes a sealant infused with a crystal powder and optionally an essential oil. The sealant can be applied to fingernails. No nail polish composition is described.

US 2012/0213718 describes a method of adding active ingredients, in an emulsion form, to a nail varnish. The nail varnish described is a system for delivering an active ingredient not a cosmetic polish. Essential oils are one of many active ingredients that can be included in the emulsion. An essential oil varnish is not exemplified in US 2012/0213718. The varnish for delivery of an active ingredient is not designed to be fragrant either before or after hardening of the varnish. Neither is the varnish designed to meet the performance characteristics of a cosmetic polish.

WO 1997/025018 described a scented nail polish comprising at least one solvent, at least one resin, at least one colorant and at least one liquid perfume wherein the weight ratio of resin to liquid perfume ranges from about 2.5:1 to about 1:2.5. The use of essential oil as the perfume is not contemplated.

WO 2013/004204 describes gel nail polish with a compound for providing a fragrance. Gel nail polish includes prepolymerised resin that is set by polymerisation under ultra violet (UV) or light-emitting diode (LED) radiance. The composition of gel nail polish is significantly different to that of traditional nail polish. The use of essential oil as a fragrance is not contemplated by WO 2012/004204.

CN 103655277 describes a cosmetic nail polish with a light fragrance that includes 7 to 14% w/w rose essential oil.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention provides a nail polish made fragrant by essential oil. The cosmetics industry has previously considered essential oil unsuitable as a fragrance for fragrant cosmetic nail polish. The stability difficulties in formulating a nail polish with an essential oil are expected to result in a product with insufficient shelf-life and/or without the qualities required of a cosmetic nail polish, such as, sufficient hardening and chip resistance. Surprisingly, the present invention discloses a stable cosmetic nail polish made fragrant with one or more essential oils and retains the features required of a cosmetic nail polish including appropriate shelf-life, setting hardness and chip resistance while allowing the essential oil fragrance to volatilise and be smelt following setting of the polish.

In one aspect, the present invention provides a stable cosmetic nail polish comprising a homogeneous mixture of one or more solvents, one or more film formers, one or more essential oils and one or more of the stabilising agents stearalkonium bentonite and stearalkonium hectorite.

Stearalkonium bentonite and stearalkonium hectorite are traditionally used as suspension agents to maintain the suspension of solid colour particles or other solids, such as, shimmer agents or glitter, in the liquid nail polish. Surprisingly, it has been found that the use of stearalkonium bentonite and stearalkonium hectorite stabilises nail polish formulations containing liquid essential oil. Without wishing to be bound by theory, it is thought that these stabilizing agents maintain the dispersion of the liquid essential oil in the polish and prevent the liquid essential oil from resulting in the separation of the other ingredients in the nail polish, thereby allowing for the preparation of stable cosmetic nail polish made fragrant by essential oil.

In one embodiment, the one or more stabilising agents are about 1% or less of the weight of the nail polish. The stabilising agents may be less than about 1% of the weight of the formulation or 0.1 to 0.9% w/w of the nail polish. The stabilising agent may also be 0.5 to 1% w/w or 0.4 to 0.8% w/w of the nail polish. The stabilising agent may be at least about 0.1% w/w, about 0.4% w/w or about 0.5% w/w of the nail polish. In preferred embodiments, the stabilising agent is stearalkonium bentonite.

In one embodiment, the essential oil comprises up to about 10% w/w of the nail polish. Alternatively, the essential oil comprises up to about 5% w/w of the nail polish or the essential oil comprises up to about 3% w/w of the nail polish. In another embodiment, the essential oil comprises at least about 2% w/w of the nail polish or, alternatively, at least about 3% w/w of the nail polish. In preferred embodiments, the essential oil comprises less than about 5% w/w of the nail polish. For clarity, the essential oil may comprise about 2% to about 10% w/w, about 2% to about 7% w/w, about 2% to about 6% w/w, about 3% to about 7% w/w, about 2% to about 5% w/w, about 3% to about 6% w/w, about 3% to about 5% w/w and about 3% to about 4% w/w of the nail polish.

In particularly preferred embodiments, the essential oil comprises about 3% w/w of the nail polish. In an alternative embodiment the essential oil comprises about 9% w/w of the nail polish. In a further embodiment the essential oil comprises about 6% w/w of the nail polish. In yet another embodiment the essential oil comprises about 4.5% w/w of the nail polish.

The essential oil may be prepared by steam distillation, cold expression or vertical steam distillation. The essential oil may be certified organic and/or therapeutic grade. Therapeutic grade essential oil is preferred because there are lower impurity levels, which result in better formulation stability.

In one embodiment of the present invention, the one or more film formers comprise 10 to 45% w/w of the nail polish. The nail polish may also have 15 to 35% w/w or 20 to 25% w/w film former. There may be 1 to 5, 1 to 4 or 1 to 3 film formers.

There may be a single film former. If there is a single film former, it is preferred that the film former is nitrocellulose. Where there is a single film former, the film former may be 10 to 30% w/w, 15 to 25% w/w or 18 to 22% w/w of the nail polish.

There may also be more than one film former. Where there is more than one film former, it is preferred that one of the film-former is nitrocellulose. Other suitable film formers include adipic acid/neopentyl glycol/trimellitic anhydride copolymer, styrene acrylates copolymer and acrylates copolymer. In one embodiment, the film formers in the nail polish comprise nitrocellulose, adipic acid/neopentyl glycol/trimellitic anhydride copolymer, styrene acrylates copolymer and acrylates copolymer.

The nail polish may have two film formers. Where the nail polish has two film formers, the film formers are preferred to be nitrocellulose and adipic acid/neopentyl glycol/trimellitic anhydride copolymer. Where nitrocellulose and adipic acid/neopentyl glycol/trimellitic anhydride copolymer are present as film formers in a nail polish with either 2 or more than 2 film-formers it is preferred if the nitrocellulose is 10 to 30% w/w, 15 to 25% w/w or 18 to 22% w/w of the nail polish and the adipic acid/neopentyl glycol/trimellitic anhydride copolymer is 3 to 10%, 4 to 8% or 5 to 6% w/w of the nail polish.

The nail polish may have 3 film formers. Where there are 3 film formers, it is preferred if the film formers are nitrocellulose, adipic acid/neopentyl glycol/trimellitic anhydride copolymer and styrene acrylates copolymer. Where there are 3 film formers, the preferred amounts of nitrocellulose and adipic acid/neopentyl glycol/trimellitic anhydride copolymer are as set out above and the preferred amount of styrene acrylates copolymer is 1% or less or less than 1%.

In one embodiment of the present invention, the one or more solvents comprise 20 to 60% w/w of the nail polish. The solvents may also be 30 to 50% w/w or 35 to 45% w/w of the nail polish. It is common for nail polish to be formulated with more than one main solvent. 2 to 3 main solvents are preferred. The nail polish may have 1 to 7, 1 to 5, 2 to 7, 2 to 5 or 2 to 4 solvents.

In preferred formulations of the invention the one or more solvents comprise ethyl acetate and butyl acetate. In these formulations, the ethyl acetate and butyl acetate may each independently be 10 to 30% w/w of the nail polish. Each of the ethyl acetate and butyl acetate may also be 15 to 25% w/w or 18 to 22% w/w of the nail polish. The ethyl acetate and butyl acetate may be present in equivalent amounts, that is, the same weight percent of the nail polish formulation as each other. However, in preferred formulations of the invention there is more ethyl acetate than butyl acetate, that is, the weight percentage of ethyl acetate is greater than the weight percentage of butyl acetate.

There may be 2 solvents in the nail polish. If there are 2 solvents in the formulation, it is preferred that the solvents are ethyl acetate and butyl acetate and that the amounts of each solvent are as described above.

There may be 3 solvents in the nail polish. If there are 3 solvents in the nail polish, it is preferred if 2 of those solvents are ethyl acetate and butyl acetate and that the third solvent is either acetyl tributyl citrate or isopropyl alcohol. The ethyl acetate and butyl acetate are preferred to be present in the amounts discussed above and the third solvent is preferred to be 3 to 20% w/w, 6 to 15% or 8 to 10% w/w of the nail polish.

There may be 4 solvents in the nail polish. If there are 4 solvents in the nail polish, it is preferred if 2 of those solvents are ethyl acetate and butyl acetate and that the other 2 solvents are acetyl tributyl citrate and isopropyl alcohol. The ethyl acetate and butyl acetate are preferred to be present in the amounts discussed above and the acetyl tributyl citrate and isopropyl alcohol are preferred to each independently be 3 to 10% w/w, 4 to 8% or 5 to 7% w/w of the nail polish.

In one embodiment, the solvents in the nail polish consist of ethyl acetate, butyl acetate, acetyl tributyl citrate, isopropyl alcohol and n-butyl alcohol. In this embodiment, the preferred quantities of each solvent are as discussed above.

In some embodiments of the present invention, the nail polish further comprises at least one of one or more plasticisers, one or more UV inhibitors, one or more thickening agents, one or more buffers. Appropriate plasticisers, UV inhibitors, thickening agents and buffers will be apparent to a nail polish formulator. Preferred plasticisers include acrylates copolymer, polyvinyl butyral and trimethyl pentanediyl dibenzoate. The plasticiser may be 1 to 5% w/w or 1 to 3% w/w of the nail polish. Acrylates copolymer is the preferred plasticiser, however, the nail polish may comprise 1 to 3 plasticisers or, alternatively, all three of the preferred plasticisers. Acrylates copolymer is preferred to be 1 to 3% w/w of the nail polish.

In one embodiment, the nail polish comprises:
10-30% ethyl acetate;
10-30% butyl acetate; and
10-30% nitrocellulose.

The above embodiment may also comprise 2 to 6, 3 to 5 or about 3% w/w essential oil. The essential oil can be certified organic and/or therapeutic grade as described elsewhere and include other ingredients as discussed elsewhere.

In preferred embodiments, the nail polish of the present invention is a 3-free nail polish that does not contain toluene, formaldehyde or dibutyl phthalate. In alternative preferred embodiments, nail polish of the present invention is a 5-free nail polish that does not contain toluene, formaldehyde, dibutyl phthalate, formaldehyde resin or camphor. It is also preferred for the nail polish to be a traditional nail enamel, that is, not a water based nail enamel, not a gel polish that is hardened by polymerisation and not an acrylic polish designed for use on acrylic nails but not suitable for use on human nails.

In one aspect, the present invention provides a stable cosmetic nail polish comprising a homogeneous mixture of one or more solvents, one or more film formers, one or more essential oils and one or more stabilising agents as described above, wherein following application of the nail polish to a nail and hardening of the nail polish, the nail polish retains an aroma from the essential oil. The aroma may be retained for at least 3 hours and up to 7 days or for at least 24 hours and up to 7 days following hardening of the nail polish. The aroma may be retained for at least 24 hours following hardening of the nail polish. Alternatively, the aroma may be retained for at least 2 days following hardening of the nail polish. Preferably, the aroma is retained for 2 to 5 days or 2 to 3 days following hardening of the nail polish. The solvents, film formers, stabilising agents, essential oil and other ingredients, such as plasticisers, may be as discussed elsewhere.

In another embodiment, the present invention provides a stable cosmetic nail polish comprising a homogeneous mixture of one or more solvents, one or more film formers, one or more essential oils and one or more stabilising agents, wherein following application of the nail polish to a nail and hardening of the nail polish, the nail polish retains an aroma from the essential oil despite ordinary water contact, such as hand washing and showering over the lengths of time discussed in the paragraph above. The solvents, film formers, stabilising agents, essential oil and other ingredients, such as plasticisers, may be as discussed elsewhere.

In one aspect, the present invention provides a stable cosmetic nail polish comprising a homogeneous mixture of one or more solvents, one or more film formers, one or more essential oils and one or more stabilising agents, wherein the nail polish remains stable following 3 months storage at about −5° C., 4° C., 25° C. or 45° C. and following the storage the nail polish retains an aroma from the essential oil following hardening of the polish on a finger or toe nail. Preferably, the nail polish remains stable following 3 months storage at each one of about −5° C., 4° C., 25° C. or 45° C. The solvents, film formers, stabilising agents, essential oil and other ingredients, such as plasticisers, may be as discussed elsewhere.

As would be clear to those skilled in the art, the nail polish is stable if there are no signs of drop out, sedimentation, precipitation, separation, syneresis or any other undesirable visual effects and the nail polish still meets the good performance criteria for a cosmetic nail polish.

The nail polish may also be stable following 6 months storage at about −5° C., 4° C., 25° C. or 45° C. Stability following 6 months storage at 25° C. is most reflective of usual storage conditions. Preferably, the nail polish remains stable following 6 months storage at each one of about −5° C., 4° C., 25° C. or 45° C.

In preferred embodiments, the nail polish remains stable following 12 months storage at about −5° C., 4° C., 25° C. or 45° C. Stability following 12 months storage at 25° C. is preferred. Preferably, the nail polish remains stable following 12 months storage at each one of about −5° C., 4° C., 25° C. or 45° C.

In addition, following the storage the nail polish for 3 months, 6 months or 12 months as discussed above, the nail polish may be applied to a nail and allowed to harden and following hardening retain an aroma either immediately or both immediately and after the passage of time (ie the aroma is retained for hours or days after application) as discussed elsewhere and/or retains an aroma despite ordinary water contact (as discussed elsewhere).

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### DETAILED DESCRIPTION OF THE EMBODIMENTS

It will be understood that various terms employed in the specification, examples and claims have meanings that will be understood by one of ordinary skill in the art. However, certain terms are defined below.

As used herein, except where the context requires otherwise, the term “comprise” and variations of the term, such as “comprising”, “comprises” and “comprised”, are not intended to exclude further additives, components, integers or steps.

The term “natural ingredient” as used throughout the specification is understood to mean an ingredient from a natural source (eg a plant) that has only been subject to physical processing without chemical reaction, for example, the ingredient may have been washed, distilled, ground, separated and/or concentrated by physical means to produce material that is identifiable in the original source material.

The term “certified organic” as used throughout the specification is understood to mean certified under the Australian Certified Organic Standard, the United States Department of Agriculture's National Organic Program (NOP), the European Ecocert Organic Standard or a similar standard authorised by another country or region.

The term “cosmetic standard(s)” as used throughout the specification is understood to mean ingredients that meet the Australian National Industrial Chemicals Notification and Assessment Scheme (NICNAS) standards, the standards of the European Commission on cosmetic substances and ingredients as set out in the CosIng database, the standards set by the United States Food and Drug Administration (FDA) on cosmetics and personal care products or a similar standard authorised by another country or region.

The phrase “cosmetic nail polish” as used throughout the specification means a non-medical nail polish that, when applied to a nail, does not damage the nail and a nail polish that has the properties of a cosmetic nail polish, that is, even distribution, good hardness etc.

The term “aroma” as used throughout the specification is understood to mean an aroma that can be smelt from at least 10 cm from the nose.

The term “essential oil” as used through the specification is understood to mean a concentration of the volatile aromatic compounds of one or more plants. Essential oils generally contain one or more terpenes, esters, aldehydes, ketones, alcohols, phenols and oxides. Essential oils are not technically oils as they are not composed of lipids. The essential oil can be from plant leaves or flowers etc. Essential oils are different to carrier oils, which are lipids that are generally pressed from the fatty portions of plants, for example, seeds. In aromatherapy, essential oils are diluted into carrier oils before application to the skin. Generally, essential oils are used for scent or therapeutic effect and carrier oils for their moisturising properties.

The term “therapeutic grade essential oil” as used throughout the specification is understood to mean an essential oil that meets the requirements for International Standards Organization (ISO) certification, in other words, the essential oil meets the ISO adopted standards prepared by The Association French Normalization Organization Regulation (AFNOR standards) for certification for essential oils.

Some conventional nail polish ingredients are toxic and/or irritants. There has been a shift in the market towards avoiding certain ingredients by the formulation of either “3-free” or “5-free” formulas. A 3-free formula does not contain toluene, formaldehyde or dibutyl phthalate (DBP). A 5-free formula does not contain toluene, formaldehyde, DBP, formaldehyde resin or camphor.

The solvent(s) function to maintain the nail polish as a liquid until application of the nail polish to a nail. Upon application of the nail polish to a nail the solvents volatilize allowing the nail polish film formers to harden. A nail polish may have one or more main solvents and a number of minor solvents. Combinations of 3 or more solvents are preferred because when multiple solvents are used each solvent is less likely to interact poorly with the essential oil resulting in instability in the formulation.

A suitable UV inhibitor is benzophenone. A suitable buffering agent is phosphoric acid. A suitable thickening agent is silica.

The nail polish of the invention may be coloured or transparent, eg, a clear top or base coat. When the nail polish is coloured, the nail polish further comprises one or more colours and/or colour effects such as shimmer, pearlescent, glitter, metallic, high luster, mattifying or opacifying effects.

Other suitable ingredients will be evident to a nail polish formulator.

In one embodiment, the nail polish comprises:
20-60% w/w solvent
10-45% w/w film former
up to 1% w/w stabilising agent
2-10% w/w essential oil

Preferably, the above embodiment has 2-6%, 2-5% or 3-5% w/w essential oil.

In another embodiment, the nail polish comprises:
10-30% ethyl acetate
10-30% butyl acetate
10-30% nitrocellulose
3-10% adipic acid/neopentyl glycol/trimellitic anhydride copolymer
up to 1% stearalkonium bentonite
2-10% w/w essential oil

Preferably, the above embodiment has 2-6%, 2-5% or 3-5% w/w essential oil.

Traditional essential Oils are aromatic oils extracted from plant materials such as flowers, leaves, bark, roots, and tree resins. There are many different ways to extract essential oils from plants including, for example steam distillation, solvent extraction, and carbon dioxide extraction (super critical fluid extraction).

Solvent extraction is the method most commonly used by the perfume industry but solvent extraction does not produce therapeutic grade essential oils. Solvents such as hexane, acetone etc are used in the process increasing the impurity levels in the essential oil.

Distilling essential oils by steam distillation can be accomplished three different ways. They are:
Water Distillation;
Water/Steam Distillation; and
Straight Steam.

Water distillation involves placing the plant material in boiling water, capturing the water and separating out the essential oil. Water/Steam Distillation includes pushing steam and water around and though the plant material, then capturing the water and steam and separating out the essential oil. The straight Steam Distillation Method involves pushing steam through the plant material and then collecting the essential oil from the steam. In all methods, steam is used to rupture the oil membranes in the plant and release the volatile essential oil. The steam carries the essential oil to a condenser and then as the steam re-liquefies the lighter essential oil floats on top of the water.

Preferably, the essential oils are made from over 90% natural ingredients, more preferably 100% natural ingredients. The essential oils may be certified organic.

There are therapeutic grades of essential oils. Only essential oils prepared by steam distillation or cold expression can be therapeutic grade. There are also purity requirements for therapeutic grade essential oils. These requirements are set out by International Standards Organization (ISO), which adopted the standard prepared by The Association French Normalization Organization Regulation (AFNOR standards) for certification for essential oils. ISO certification indicates an essential oil has the chemical constituents supposed to be in a therapeutic grade of that essential oil. The temperature, pressure and equipment used in preparation of the essential oil all affect the quality of the oil and whether or not it will meet ISO certification standards.

Some non-therapeutic grade essential oils contain contaminants that result in chemical instability in a nail polish, for example, contaminants can cause discolouration of the polish or result in physical instability, for example, separation from the polish. Thus, it is preferred for the essential oil to be therapeutic grade according to the ISO standards.

The fragrance of many essential oils is known to have a calming and/or uplifting effect on the body and mood.

Suitable essential oils include peppermint oil, ylang ylang oil, lavender oil, lime oil, neroli oil, sweet orange oil, cedarwood atlas oil and combinations thereof. The essential oil is preferred to be natural and especially preferred to be certified organic. The essential oil may be therapeutic grade. The use of therapeutic grade essential oil does not necessarily make the nail polish therapeutic.

Essential oils are volatile ingredients and can degrade and/or evaporate if exposed to heat. It is recommended that the essential oil be the last ingredient added to the nail polish during formulation, after the formulation has cooled and after the colour (if any) is added. Before addition of the essential oil the nail polish is prepared by established methods known to nail polish formulators. One example of a common method of formulation involves combining 75% of the solvent, any buffer and any diluent and mixing; the film former is added while stirring; the remaining solvent is added; any plasticiser is added; the remaining ingredients are added; and continuously mixed until homogeneous. However, other methods of formulation will be known to nail polish formulators.

The examples that follow are intended to illustrate but in no way limit the present invention.

### Example 1—Formulations

The following 5-free base nail polishes were prepared:

| Ingredient Details | CAS NO. | FUNCTION | % w/w |
|---|---|---|---|
| ETHYL ACETATE | 141-78-6 | Solvent | 10-30 |
| BUTYL ACETATE | 123-86-4 | Solvent | 10-30 |
| NITROCELLULOSE | 9004-70-0 | Film Former | 10-30 |
| ADIPIC ACID/NEOPENTYL GLYCOL/TRIMELLITIC | 28407-73-0 | Film former | 3-10 |
| ANHYDRIDE COPOLYMER | | | |
| ACETYL TRIBUTYL CITRATE | 77-90-7 | Solvent | 3-10 |
| ISOPROPYL ALCOHOL | 67-63-0 | Solvent | 3-10 |
| STYRENE ACRYLATES COPOLYMER | 9010-92-8 | Film former | <1 |
| STEARALKONIUM BENTONITE | 12691-60-0 | Stabilising | <1 |
| | | agent/suspension | |
| | | agent | |
| N-BUTYL ALCOHOL | 71-36-3 | Solvent | <1 |
| ACRYLATES COPOLYMER | 25035-69-2 | Plasticiser | 1-3 |
| BENZOPHENONE 1 | 131-56-6 | UV Inhibitor | <1 |
| POLYVINYL BUTYRAL | 63148-65-2 | Plasticiser | <1 |
| TRIMETHYL PENTANEDIYL DIBENZOATE | 68052-23-3 | Plasticiser | <1 |
| SILICA | 7631-86-9 | Thickening | <1 |
| | | agent | |
| POLYETHYLENE TEREPHTHALATE | 25038-59-9 | Colour Effect | <1 |
| ALUMINIUM HYDROXIDE | 21645-51-2 | Colour Effect | <1 |
| BIS | 146277-66-9 | Colour Effect | <1 |
| (GLYCIDOXYPHENYL)PROPANE/BISAMINOMETHYLN | | | |
| ORBONANE COPOLYMER | | | |
| DIETHYLAMINOMETHYLCOUMARIN | 61791-47-7 | Colour Effect | <1 |
| COCAMIDE DEA | 68603-42-9 | Colour Effect | <1 |
| CALCIUM ALUMINIUM BOROSILICATE | 65997-17-3 | Colour Effect | <1 |
| PHOSPORIC ACID | 7664-38-2 | Buffering | 0.1-0.3 |
| | | Agent | |
| MICA | 12003-38-2 | Colouring | <1 |
| | | Agent | |

The base polish is prepared by adding each ingredient listed above in the order listed above and mixing until homogeneous.

3% w/w of lavender essential oil was added to the base polish and mixed until homogeneous with the base polish. 3% w/w of peppermint essential oil was added to the base polish for a different scent. Nail polish with 3% ylang ylang oil was also prepared and nail polish with 3% w/w “beautiful blend” (a mixture of lime, neroli, sweet orange, cedarwood atlas and ylang ylang). The essential oils used were certified organic and therapeutic grade. The fragrant nail polish is prepared in 15 ml bottles.

The stabilising agent stearalkonium bentonite was used in these formulations, however, stearalkonium hectorite is also expected to work.

### Example 2—Stability Testing

Method:

1. The nail polishes of Example 1 (ie a nail polish with 3% w/w lavender essential oil, another with 3% w/w peppermint essential oil, another with 3% w/w ylang ylang essential oil and another with 3% w/w “beautiful blend” (a mixture of lime, neroli, sweet orange, cedarwood atlas and ylang ylang essential oils) were prepared in 15 ml nail polish bottles. One sample of each essential oil blend was prepared per test temperature.

2. A bottle of each fragrance was stored at each one of −5° C., 4° C., 25° C. and 45° C.

3. Each sample was weighed and stored in a location without exposure to strong light.

4. Each sample was re-weighed weekly and checked for any signs of drop out, sedimentation, precipitation, separation, syneresis or any other undesirable visual effects.

5. Testing was continued for 12 months

Each nail polish tested was stable for at least one year, that is, after one year no sediment or discolouration was visible, the nail polish still met performance criteria for a cosmetic nail polish, such as appropriate setting time and hardness etc, and the nail polish could still be applied to a nail, allowed to harden and following hardening retained an aroma. The aroma lasted despite ordinary water contact.

Essential oils are prone to oxidation. The retention of the desired aroma from the essential oil after storage for 12 months in a nail polish formulation is particularly unexpected.

### Example 3—Quantity of Essential Oil

The inventor of the present application has tested nail polish formulations that required 9%, 6%, 4.5% and 3% essential oil before the aroma of the essential oil could be detected. Without being bound by theory, it is thought that the difference in the required quantity of essential oil depends on the odour of the other ingredients of the nail polish. It is preferred for the nail polish ingredients other than the essential oil(s) to have low odour. This may be achieved by selecting low odour ingredients or by selecting high purity grades for ingredients as odour is sometimes caused by impurities.

### Example 4—Retention of Aroma

The nail polish formulations prepared in Example 1 were applied to human fingernails and allowed to harden. It was confirmed that the nail polish retained an aroma following hardening. The aroma was retained for 24 hours to 7 days following hardening of the nail polish despite ordinary water contact, for example, showering and hand washing. Most commonly often the aroma was retained for 2 to 5 days.

Without wishing to be bound by theory, the difference in the length of the retention of the aroma from application of the same nail polish to different people is thought to relate to the quality of the human nail to which the nail polish is applied. For example, a thin porous nail is likely to absorb the essential oil from the nail polish and retain its aroma for a shorter period than a thick nail.

It will be understood that the invention disclosed and defined in this specification extends to all alternative combinations of two or more of the individual features mentioned or evident from the text or drawings. All of these different combinations constitute various alternative aspects of the invention.

1. A stable cosmetic nail polish comprising a homogeneous mixture of one or more solvents, one or more resins, one or more film formers, one or more essential oils and one or more of the stabilising agents stearalkonium bentonite and stearalkonium hectorite.

2. The nail polish according to claim 1, wherein the one or more solvents comprise 20 to 60% w/w of the nail polish.

3. The nail polish according to claim 1, wherein there are 1 to 7 solvents.

4. The nail polish according to claim 1, wherein the one or more film formers comprise 10 to 45% w/w of the nail polish.

5. The nail polish according to claim 1, wherein the nail polish further comprises one or more plasticisers.

6. The nail polish according to claim 5 wherein the one or more plasticisers is 1 to 5% w/w of the nail polish.

7. A stable cosmetic nail polish comprising a homogeneous mixture of one or more solvents, one or more resins, one or more film formers, one or more essential oils and one or more of the stabilising agents stearalkonium bentonite and stearalkonium hectorite, wherein the nail polish comprises:
10-30% ethyl acetate;
10-30% butyl acetate; and
10-30% nitrocellulose.

8. The nail polish according to claim 7, wherein the stabilising agent is about 1% w/w or less of the nail polish.

9. The nail polish according to claim 7, wherein the nail polish comprises up to about 10% w/w essential oil.

10. The nail polish according to claim 7, wherein the nail polish comprises 2 to 6% w/w essential oil.

11. The nail polish according to claim 7, wherein following application of the nail polish to a nail and hardening of the nail polish, the nail polish retains an aroma from the essential oil.

12. The nail polish according to claim 11, wherein the nail polish retains an aroma from the essential oil for at least 24 hours following application of the nail polish to a nail and hardening of the nail polish.

13. The nail polish according to claim 11, wherein the nail polish retains an aroma following application of the nail polish to a nail and hardening of the nail polish despite ordinary water contact.

14. The nail polish according to claim 7, wherein the nail polish is stable following 12 months storage at 25° C.

15. A stable cosmetic nail polish comprising a homogeneous mixture of:
10-30% ethyl acetate
10-30% butyl acetate
10-30% nitrocellulose
up to 1% stearalkonium bentonite
2-10% w/w essential oil.
